(19) United States
(12) Patent Application Publication
(10) Pub. No.: US 2011/0066226 A1
(43) Pub. Date: Mar. 17, 2011

(54) Implantable Venous Valve for Treatment of Erectile Dysfunction

(75) Inventors: Rodney Bell, Monivea (IE); Sean Ward, Dublin (IE)

(73) Assignee: Medtronic Vascular, Inc., Santa Rosa, CA (US)

(21) Appl. No.: 12/559,618

(22) Filed: Sep. 15, 2009

(51) Int. Cl. A61F 5/41; A61F 2/06

(52) U.S. Cl. 623/1.24; 600/38

(57) ABSTRACT

A venous valve is disclosed for implantation in and selectively restricting the outflow of blood from a penile vein to aid a user in achieving and/or maintaining an erection. The venous valve includes a self-expanding stent framework defining a blood flow lumen there through. The self-expanding stent framework is constructed to recoil from a radially compressed configuration in which the blood flow lumen is narrowed to restrict blood flow through the venous valve to a radially expanded configuration in which the blood flow lumen is fully open to permit unrestricted blood flow through the venous valve. A recoil delay component is attached to the self-expanding stent framework for slowing the recoil of the self-expanding stent framework to thereby provide an extended time period during which the blood flow lumen is narrowed and blood flow through the venous valve is restricted.

### FIELD OF THE INVENTION

The present invention relates to an intravascular device for treatment of erectile dysfunction. More particularly, the intravascular device is a venous valve that may be implanted in a penile vein to selectively restrict blood outflow through the penile vein in order to achieve and/or maintain an erection.

### BACKGROUND OF THE INVENTION

The National Institutes of Health estimates that 30 million American men suffer from mild, moderate or complete erectile dysfunction. Erectile dysfunction is the chronic, i.e., greater than three months duration, inability to maintain a penile erection for satisfactory sexual intercourse.

There are both psychological and physical causes of erectile dysfunction. Most causes of erectile dysfunction have an adverse effect on nerves and/or blood vessels to, from, and within the penis. Vascular disease is considered a leading physical cause of erectile dysfunction with atherosclerosis of the penile arteries alone accounting for about 40% of patients over 40 years of age with erectile dysfunction. Other possible vascular-related causes include diabetes, hypertension, high cholesterol, renal disease, and smoking.

Essentially, penile erection occurs when the two corpora cavernosa fill with blood and maintain pressure adequate for penetration. Each corpus cavernosum is fed by a deep artery of the penis located in the center of each cavernosum. Each deep artery has many smaller coil-shaped arteries, called helicine arteries extending downstream therefrom that open directly into the corpora cavernosa. Erection of the penis is a parasympathetic nervous system process that effects the release of neurotransmitters, which allows the relaxation of smooth muscle fibers surrounding the helicine arteries resulting in an increase in arterial inflow in the corpora cavernosa. Blood then fills these erectile compartments, and in the process compresses the penile veins that drain these tissues. The obstruction of venous flow is as important in obtaining and maintaining an erection as is an adequate arterial blood supply. The net effect of this increased inflow and decreased blood outflow is to raise the pressure of the corpora cavernosa to approximately the mean arterial pressure of the cavernosal artery, which in a normal patient is approximately 100 mm Hg. Subsequent activation of the sympathetic nervous system returns the penis to a flaccid state. With reference to FIG. 1 that depicts a sectional view of a portion of a penis, major venous drainage of the penis occurs through the deep dorsal vein 100 that returns blood from the shaft or pendulous portion of the cavernosa as well as from the glans penis. In addition to a few other penile veins, such as the cavernosal veins, blood also exits the penis via the superficial dorsal vein 102.

In cases where erectile dysfunction occurs due to, or is complicated by venous leakage there are various bands, rings and ligatures that have been suggested to restrict blood flow leaving the penis and thereby enable a patient to achieve an erection. Some such restrictive devices are externally secured around the base of the penis and are worn only during sexual activity, whereas others are surgically or laparoscopically implanted and are externally activated to temporarily contract around penile tissue or exit veins of the penis to enable the erection. A binary duct valve has also been suggested for use in penile veins to selectively induce tumescence. The binary duct valve is implanted surgically or via a needle puncture into the vein and includes a ball valve of a magnetic material that is operated extracorporeally by a user manipulating a magnet. Each of the afore-mentioned apparatuses for treating erectile dysfunction that is caused or aggravated by venous leakage suffers from disadvantages, some of which are addressed by a venous valve according to the present invention.

### BRIEF SUMMARY OF THE INVENTION

Embodiments hereof are directed to an implantable venous valve for selectively restricting the outflow of blood from a penile vein to aid a user in achieving and/or maintaining an erection. The venous valve includes a self-expanding stent framework defining a blood flow lumen therethrough. The self-expanding stent framework is constructed to recoil from a radially compressed configuration in which the blood flow lumen is narrowed to restrict blood flow through the venous valve to a radially expanded configuration in which the blood flow lumen is fully open to permit unrestricted blood flow through the venous valve. A recoil delay component is attached to the self-expanding stent framework for slowing the recoil of the self-expanding stent framework and thereby provides an extended time period during which the blood flow lumen is narrowed such that blood flow through the venous valve is restricted.

Embodiments hereof are also directed to methods of using a venous valve for selectively restricting the outflow of blood from a penile vein to aid in achieving and/or maintaining an erection. The methods include implanting a venous valve into a penile vein at a target location that is susceptible to a compressive radial force exerted on the penis. A venous valve for use in methods hereof includes a self-expanding stent framework defining a blood flow lumen there through and a recoil delay component attached to the self-expanding stent framework that delays the recoil of the self-expanding stent framework from a radial compressed configuration to a radially expanded configuration. The methods further include firmly pressing on the penis to impart a compressive radial force on the venous valve implanted at the target location thereby radially compressing the self-expanding stent framework and initiating an extended time period during which the blood flow lumen is narrowed to restrict blood outflow from the penile vein.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a partial sectional view of a portion of a penis.

FIG. 2 is a perspective view of a venous valve in accordance with an embodiment hereof

FIG. 2A is a transverse cross-sectional view of the venous valve of FIG. 2 taken along line A-A shown in a radially expanded or open configuration.

FIG. 2B is the venous valve cross-section of FIG. 2A shown in a radially compressed or closed configuration.

FIG. 3 is a perspective view of a venous valve in accordance with another embodiment hereof.

FIG. 3A is a transverse cross-sectional view of the venous valve of FIG. 3 taken along line A-A shown in a radially expanded or open configuration.

FIG. 4 is a perspective view of a venous valve in accordance with another embodiment hereof.

FIG. 4A is a transverse cross-sectional view of the venous valve of FIG. 4 taken along line A-A shown in a radially expanded or open configuration.

FIG. 5 is a longitudinal sectional view of an alternate embodiment of the venous valve shown in FIG. 2 deployed within a penile vein.

FIG. 6 is a longitudinal sectional view of the venous valve of FIG. 5 radially compressed within the penile vein to restrict blood flow there through.

FIG. 7 is a perspective view of a catheter-based delivery system having a distal portion shown in partial section to expose a venous valve in accordance with an embodiment hereof loaded therein.

FIG. 8 is a longitudinal sectional view of the venous valve shown in FIG. 5 being deployed in a penile vein by the catheter-based delivery system shown in FIG. 7.

FIG. 9 is a longitudinal sectional view of the venous valve shown in FIG. 5 being expanded by a balloon upon initial deployment within the penile vein.

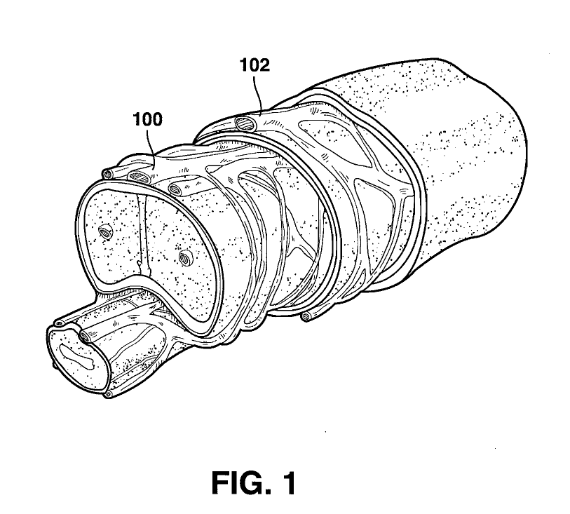

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the clinical application and uses of the invention. Although the description of the invention is in the context of placement within a blood vessel such as the superficial and deep dorsal veins, the invention may also be used in any other body passageways where it is deemed useful. Furthermore, there is no intention to be bound by any expressed or implied theory presented in the preceding technical field, background, brief summary or the following

### DETAILED DESCRIPTION

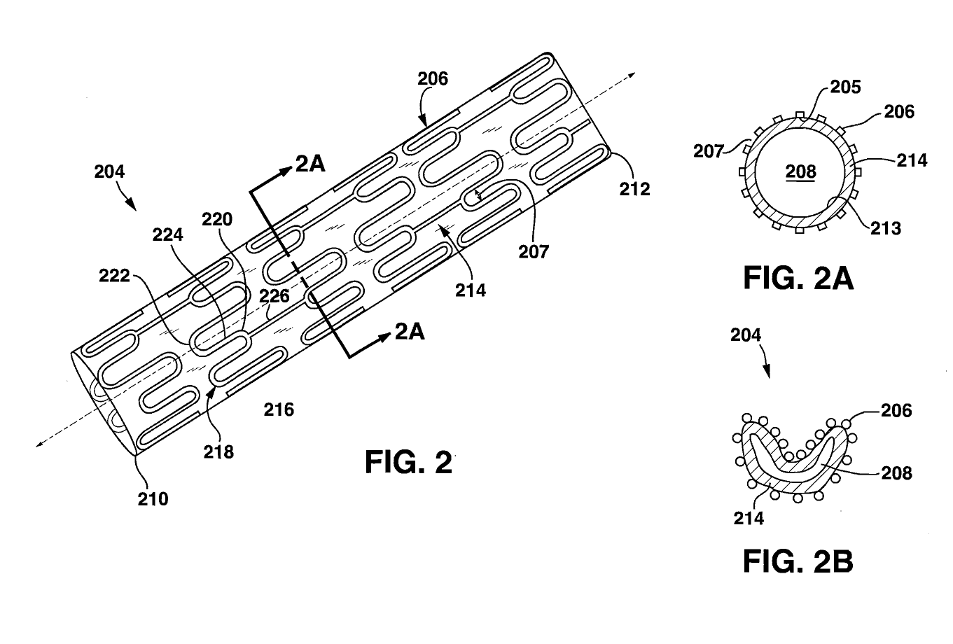

FIG. 2 is a perspective view of an implantable venous valve 204 according to an embodiment hereof with FIG. 2A being a cross-sectional view taken along line A-A. Venous valve 204 includes a self-expanding stent framework 206 that defines a blood flow lumen 208 between a first end 210 and a second end 212 thereof. A recoil delay component 214, which will be further described below, is attached to an inner surface 205 of stent framework 206 and covers interstitial spaces or openings 207 of stent framework 206, such that an interior surface 213 of recoil delay component 214 outlines blood flow lumen 208. Recoil delay component 214 is a tubular construction that is attached to the stent framework by either thermal or adhesive bonding.

Stent framework 206 is an exemplary stent framework in accordance with an embodiment of the present invention that is made self-expanding by virtue of the internal restoring forces of the spring-type or superelastic material selected for its construction. In an embodiment hereof, stent framework 206 is formed of a pseudoelastic or stress induced martensitic (SIM) alloy of nickel-titanium (nitinol). Stent framework 206 is a patterned tubular device that includes a plurality of radially expandable cylindrical rings 216. Cylindrical rings 216 are formed from struts 218 having a generally sinusoidal pattern that includes peaks 220, valleys 222, and generally straight segments 224 connecting peaks 220 and valleys 222. Connecting links 226 connect adjacent cylindrical rings 216 together. In FIG. 2, connecting links 226 are shown as generally straight links connecting a peak 220 of one ring 216 to a valley 222 of an adjacent ring 216. However, connecting links 226 may connect a peak of one ring to a peak of an adjacent ring, or a valley to a valley, or a straight segment to a straight segment. Further, connecting links 226 may be curved. Connecting links 226 may also be excluded, with a peak or valley of one ring being directly attached to a valley or a peak of an adjacent ring, such as by welding soldering, or the manner in which stent framework 206 is formed, for e.g., by etching the pattern from a flat sheet or a tube. It will be appreciated by those of ordinary skill in the art that stent framework 206 of FIG. 2 is merely an exemplary stent framework and that self-expanding stent frameworks of various forms and methods of fabrication can be used in accordance with various embodiments of the present invention. Stent framework 206 may have any stent configuration or design known in the art. Some examples of stent configurations that are suitable for use in embodiments of the present invention are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau, and U.S. Pat. No. 6,730,116 to Wolinsky et al., each of which is incorporated by reference herein in its entirety.

The superelastic or pseudoelastic material selected for forming self-expanding stent framework 206 permits venous valve 204 to recoil or recover from a radially compressed configuration in which blood flow lumen 208 is narrowed, as shown in FIG. 2B, and return to a radially expanded configuration in which blood flow lumen 208 is fully open, as shown in FIG. 2A. As used herein, the radially compressed configuration of a venous valve in accordance herewith does not mean the radial compression of the venous valve is uniform or uniformly applied about a circumference of the venous valve but instead means that the venous valve has been flattened or otherwise distorted by lateral or transverse compression that may be applied by a user pressing on one side of the venous valve thereby radially compressing the venous valve as discussed further below. As such radial compression of a venous valve in accordance herewith may result in the venous valve having an asymmetrical cross-section in the radially compressed configuration and/or may occur along only a portion of the length of the venous valve.

The recoil or recovery of stent framework 206 alone, i.e., without recoil delay component 214 attached thereto, from a radially compressed configuration to a radially expanded configuration conventionally occurs immediately upon removal of the external force causing the compression, which means that stent framework 206 conventionally will quickly or over a short time period such as 0.1-1.0 seconds, return to its radially expanded configuration. In order to slow or delay the recoil of self-expanding stent framework 206 after release of an external force in accordance with an embodiment hereof, recoil delay component 214 is coupled to self-expanding stent framework 206 to increase or extend a time period after release of an external force during which venous valve 204 is in a radially compressed configuration and blood flow lumen 208 is narrowed or flattened. Recoil delay component 214 is formed of a viscoelastic polymeric material having a thickness of 0.1-1.0 mm that exhibits slow elastic recovery in order to impart a damping effect on self-expanding stent framework 206 and that is biostable such that recoil delay component 214 will not biodegrade or bioabsorb during prolonged implantation in vivo. In another embodiment, recoil delay component 214 may be formed of a biostable elastomeric material, such as biostable polyurethane elastomers or silicone foams. The damping effect of recoil delay component 214 is expected to inhibit or delay the internal restoring forces of stent framework 206 from quickly returning venous valve 204 to a radially expanded configuration upon release of an external force. The viscoelastic polymeric material selected for recoil delay component 214 undergoes time dependent strain and therefore takes a longer period of time to elastically recover from an applied external force than does self-expanding stent framework 206 formed from a superelastic material.

In an embodiment, recoil delay component 214 should sufficiently slow the recovery of stent framework 206 from the radially compressed configuration such that blood flow lumen 208 of venous valve 204 will be narrowed or closed for a time period of between 20 to 60 minutes. When venous valve 204 is positioned in vivo within a penile vein, such as the deep dorsal vein or the superficial dorsal vein as further discussed below, blood flow through venous valve 204 may be restricted for 20 to 60 minutes so that outflow of blood from the penile vein is diminished for this time period thereby aiding the user in achieving and/or maintaining an erection. The end of the restricted time period may be defined as the time at which venous valve 204 has completely reverted to the radially expanded configuration shown in FIG. 2A. Recoil delay component 214 is expected to provide a gradual damping effect that takes place over the restricted time period such that venous valve 204 is substantially closed at the beginning of the restricted time period, completely open at the end of the restricted time period, and partially closed during the restricted time period.

Viscoelastic polymeric materials that may be adapted for use in forming recoil delay components in accordance with embodiments hereof include but are not limited to foam rubber such as foam polyurethane sold under the trademark PPT and available from Langer Biomechanics, Deer Park, N.Y., and thermoset polyether-based polyurethane material sold under the trademark SORBOTHANE, available from Sorbothane, Inc. of Kent, Ohio, and acrylate polymer sold under the trademark 3M Viscoelastic Damping Polymer 242NR02 available from 3M Corporation of St. Paul, Minn. As well, viscoelastic polymeric gels, thermoset polyurethane gels, cohesive polymeric silicone gels sold under trademarks MEMORY GEL and COHESIL available from Mentor Corporation, Santa Barbara, Calif., and slow elastic recovery hydrogels, such as hydrogels disclosed in U.S. Pat. No. 4,452,776 to Refojo which is incorporated by reference herein in its entirety, may be adapted for use in embodiments hereof.

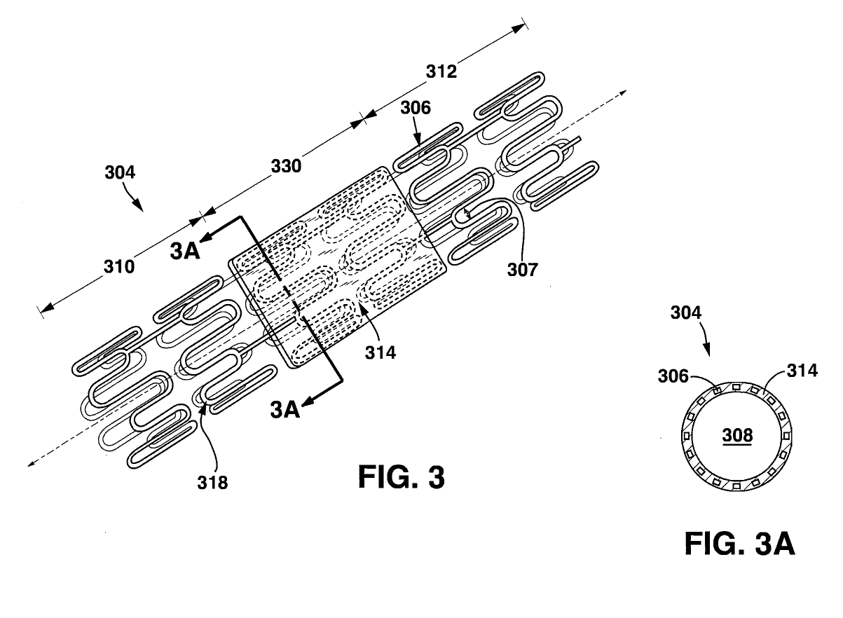

In the embodiment of venous valve 204 shown in FIG. 2, recoil delay component 214 is shown extending within stent framework 206 over its entire length from first end 210 to second end 212. In another embodiment shown in FIG. 3, venous valve 304 includes recoil delay component 314 that extends along only a middle or intermediate section 330 of self-expanding stent framework 306. Self-expanding stent framework 306 is formed of a superelastic material and has the construction described above with reference to the embodiment of FIG. 2. However in the embodiment of FIG. 3, a first end section 310 and a second end section 312 of stent framework 306 remain bare or uncovered by recoil delay component 330 and therefore sections 310, 312 are not encumbered or delayed by recoil delay component 314 when recovering from a radially compressive external force. Accordingly, first and second end sections 310, 312 of stent framework 306 immediately recoil from a radially compressed configuration to a radially expanded configuration upon removal of the external force whereas intermediate section 330 of self-expanding stent framework 306 that includes recoil delay component 314 recoils over an extended period of time, such as between 20 and 60 minutes as described above with reference to the embodiment of FIG. 2. In addition, when venous valve 304 is delivered to a delivery site within a penile vein, first and second end sections 310, 312 should readily deploy into contact with a wall of the vessel upon delivery and allow intimal growth around bare struts 318 in end sections 310, 312 after implantation.

FIG. 3A is a cross-sectional view of venous valve 304 of FIG. 3 taken along line A-A showing a fully open blood flow lumen 308. In venous valve 304 intermediate section 330 of stent framework 306 is enclosed within recoil delay component 314 such that struts 318 and interstitial spaces 307 of intermediate section 330 are completely covered by the material of recoil delay component 314. In an embodiment, venous valve 304 may be formed by over-molding recoil delay component 314 onto intermediate section 330 of stent framework 306. In another embodiment, venous valve 304 may be formed by positioning intermediate section 330 of stent framework 306 between two tubes or layers of the material that forms recoil delay component 314 and then heat bonding the tubes or layers of material together to sandwich stent framework 306 therebetween. Although intermediate section 330 and first and second end sections 310, 312 are shown to be of approximately equal lengths in the embodiment of FIG. 3, i.e., each extending for approximately a third of the length of venous valve 304, it should be understood that they may be of unequal lengths with intermediate section 330 being longer than either or both of first and second end sections 310, 312.

Recoil delay component 314 of venous valve 304 may be formed of any of the slow elastic recovery materials disclosed above with reference to recoil delay component 214 and may have a thickness of 0.1-1.0 mm in order to impart a damping effect on intermediate section 330 of self-expanding stent framework 306. The damping effect of recoil delay component 314 inhibits or delays the internal restoring forces of intermediate section 330 of stent framework 306 from quickly returning that portion of venous valve 304 from a radially compressed configuration to a radially expanded configuration upon release of an external force causing the compression. The delayed recoil of intermediate section 330 of venous valve 304 is expected to provide a time period during which blood flow lumen 308 is narrowed or closed and blood flow through venous valve 304 is restricted, such as a time period of between 20 and 60 minutes.

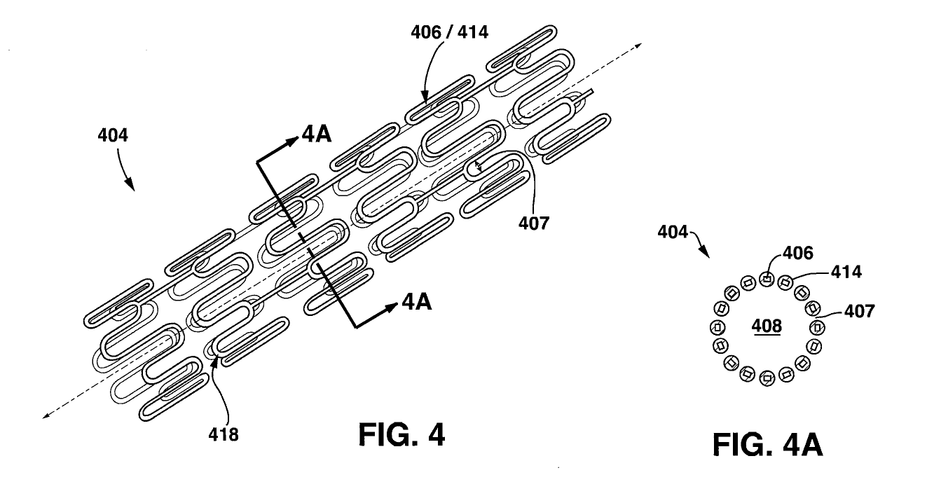

FIG. 4 is a perspective view of venous valve 404 in accordance with another embodiment hereof with FIG. 4A being a cross-sectional view of venous valve 404 taken along line A-A of FIG. 4. Venous valve 404 has a self-expanding stent framework 406 and a recoil delay component 414 that covers only struts 418 of stent framework 406. The interstitial spaces 407 between struts 418 are open along the length of venous valve 404 to permit intimal growth therein after implantation. As in the previous embodiments, self-expanding stent framework 406 is formed of a superelastic material and has the construction described above with reference to the embodiment of FIG. 2. Venous valve 404 may be formed by over-molding recoil delay component 414 only onto struts 418 of stent framework 406 or by dipping struts 418 of stent framework 406 into a solution of a viscoelastic polymeric material to form recoil delay component 414 thereon. As in the embodiment of FIG. 3, stent framework 406 of venous valve 404 may have recoil delay component 414 covering only an intermediate section thereof so that first and second end sections thereof are left bare.

Recoil delay component 414 of venous valve 404 may be formed of any of the slow elastic recovery materials disclosed above with reference to recoil delay component 214 and may have a thickness of 0.1-1.0 mm in order to impart a damping effect on self-expanding stent framework 406. The damping effect of recoil delay component 414 inhibits or delays the internal restoring forces of stent framework 406 from quickly returning venous valve 404 from a radially compressed configuration to a radially expanded configuration upon release of an external force causing the compression. The delayed recoil of venous valve 404 is expected to provide a time period during which blood flow lumen 408 is narrowed or closed and blood flow through venous valve 404 is restricted, such as a time period of between 20 and 60 minutes.

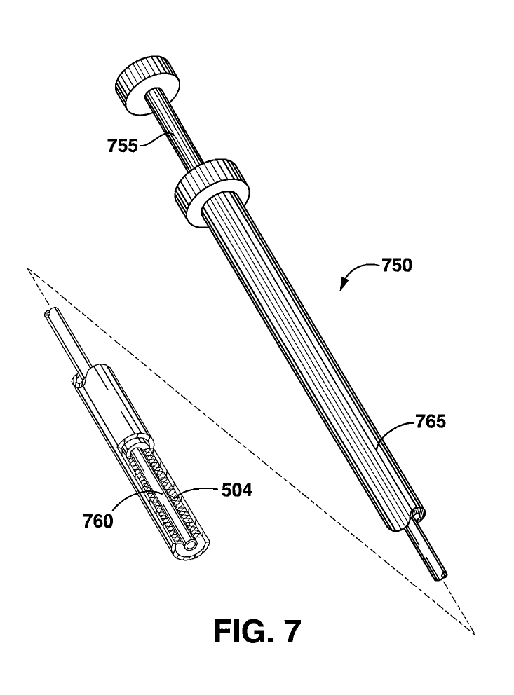

With reference to FIG. 7, deployment of a venous valve 504, which is described below, may be accomplished by tracking a catheter-based delivery system 750 through the vasculature of the patient until the venous valve is located within a target vessel, such as deep dorsal vein 100 or superficial dorsal vein 102 or other palpable penile vein. An exemplary route for tracking the catheter-based delivery system through the vasculature may include introducing the delivery system into the femoral vein and directing the system through the internal iliac vein and the internal pudendal vein to the delivery site in the target penile vein.

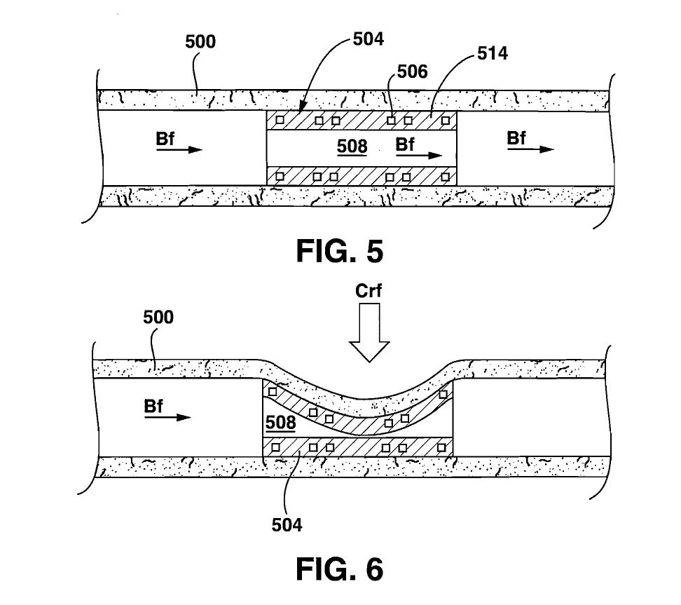

Catheter-based delivery system 750 includes an inner shaft 755 having venous valve 504 mounted around a distal end 760 thereof, and a retractable outer sheath 765 that covers and constrains venous valve 504 in a reduced diameter while delivery system 750 is tracked through a vessel to the delivery site. The operation and structure of catheter-based delivery system 750 is more fully described in U.S. Pat. No. 6,126,685 to Lenker et al., which is incorporated by reference herein in its entirety. In other embodiments, delivery systems that are well known in the art may be used to deliver implantable venous valves in accordance herewith. In embodiments hereof, the delivery site for the venous valve may be within deep dorsal vein 100 or superficial dorsal vein 102 at a location near the base of the penis proximate the point where the penile vein enters the torso. The venous valve is intended to be positioned such that it may be radially compressed within the penile vein by a user pressing firmly on the base of the penis, as discussed further below with reference to the embodiment shown in FIGS. 5 and 6.

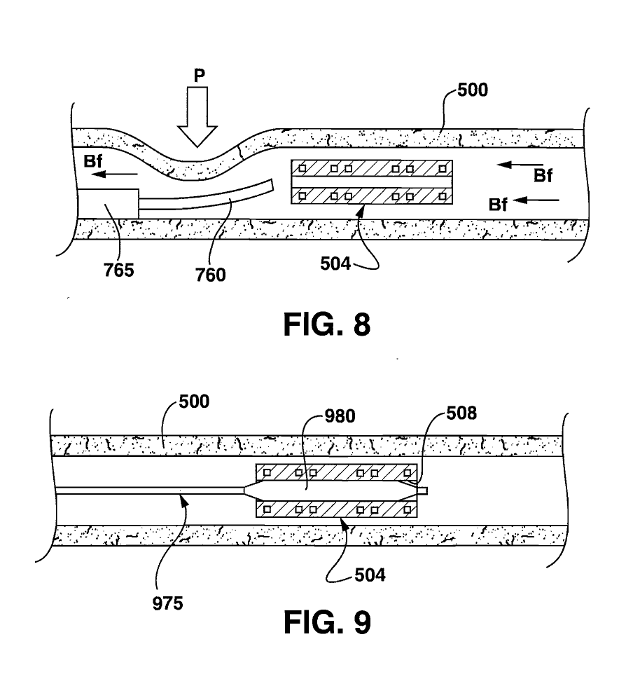

As prophetically illustrated in FIG. 8, once venous valve 504 is properly positioned within the penile vein 500, outer sheath 765 of catheter-based delivery system 750 may be retracted to release venous valve 504 so that venous valve 504 may expand into apposition with the vessel wall of the penile vein. Catheter-based delivery system 750 is then withdrawn from venous valve 504. In order to accommodate the elastic recovery of venous valve 504 being delayed upon initial deployment from the sheath, means for keeping the venous valve from moving out of the intended location while the venous valve achieves a fully expanded diameter, i.e., its radially expanded configuration, may be implemented. In one means for keeping the venous valve at the target location, venous valve 504 may include barb-like projections (not shown) that engage the vessel wall to hold the venous valve stationary while the venous valve slowly expands to its full radially expanded configuration.

Another means for keeping the venous valve 504 at the target location is shown in FIG. 8. A compression bandage or external clamp/ring (not shown) may be placed temporarily around the penis to hold the venous valve in place in the penile vein while the venous valve slowly transforms to its full radially expanded configuration. The external clamp/ring may be placed adjacent to and downstream of venous valve 504 to partially pinch off penile vein 500, as represented by arrow P, to block valve 504 from being displaced from the intended implantation site by the flow of blood, represented by arrows B_{f}. The external clamp/ring may be applied before venous valve 504 is released from catheter-based delivery system 750.

In an embodiment shown prophetically in FIG. 9, a post deployment expansion of venous valve 504 may be performed, such as by a balloon catheter 975, to radially expand venous valve 504 into apposition with a wall of a penile vein 500. In such an embodiment, delivery system 750 is modified by replacing inner shaft 755 with balloon catheter 975. Venous valve 504 is mounted in a radially compressed configuration about deflated balloon 980 and outer sheath 765 covers and constrains venous valve 504 in a reduced diameter while delivery system 750 is tracked through a vessel to the delivery site. As described above, once venous valve 504 is properly positioned within the penile vein, outer sheath 765 of catheter-based delivery system 750 may be retracted to uncover venous valve 504. Then, balloon 980 of catheter 975 is pressurized to hold venous valve 504 at the target location and upon continued inflation of balloon 980 to expand venous valve 504 into its full radially expanded configuration within penile vein 500. Balloon 980 is a soft elastomeric material to avoid damage to the recoil delay component of venous valve 504 that may be caused by expansion that is too rapid or with too much expansion force. For the embodiment shown in FIG. 3, there is no need to use a balloon to expand venous valve 304 because, as described above, first and second end sections 310, 312 of stent framework 306 immediately recoil from a radially compressed configuration to a radially expanded configuration that engages the inner wall of the penile vein upon removal of the external force. Besides the soft elastomeric balloon material, catheter 975 may have any catheter configuration or design known in the art, for e.g., dilatation catheters disclosed in U.S. Pat. No. 5,827,225 to Ma Schwab and U.S. Pat. No. 7,297,134 to Krivoruchko, each of which is incorporated by reference herein in its entirety, may be used in embodiments hereof.

Another method of delivering a venous valve in accordance with embodiments hereof may include identifying a target penile vein via ultrasound or color Doppler imaging and gaining access to the penile vein by performing a micropuncture procedure on the penis with a cannula. The venous valve may then be deployed within the penile vein through the cannula.

After initial deployment within the target penile vein, a venous valve in accordance with embodiments hereof is expected to become attached to or embedded within the penile vein due to endothelialization that occurs as cells grow around the stent framework of the venous valve. Implantable venous valves in accordance with embodiments hereof must be sufficiently endothelialized in order to prevent dislodgment from the penile vein when radially compressed and in order to “pull” the walls of the vein inward upon being radially compressed to stop or restrict the blood flow there through. Full endothelialization of venous valves in accordance herewith may occur as quickly as three weeks or may take up to eight weeks. In order to allow endothelialization of the venous valve, each of the embodiments of FIGS. 3 and 4 include bare struts and/or interstitial openings through which cell growth may occur. In addition, the recoil delay components used in embodiments hereof may be made porous in order to permit cell growth therein.

FIGS. 5 and 6 show venous valve 504 prophetically deployed and endothelialized within a target penile vein 500. Venous valve 504 is substantially similar to venous valve 204 of FIG. 2 with the difference being that stent framework 506 is completely enclosed by recoil delay component 514 for the length of venous valve 504 and recoil delay component 514 is porous to permit endothelialization to occur therein. Venous valve 504 is in a radially expanded configuration in FIG. 5 with blood flow lumen 508 fully open to permit unrestricted blood flow, represented by arrows B_{f}, through venous valve 504. Although in the expanded configuration venous valve 504 may by its very presence in the penile vein 500 interfere with blood flow, venous valve 504 in the expanded configuration is not expected to interfere with blood outflow from the penis through penile vein 500 in a manner which would be considered clinically relevant. In order to prevent or restrict blood outflow from the penile vein to aid in attaining and/or maintaining a penile erection, the user applies a compressive radial force, represented by arrow C_{rf}, to the penis that is sufficient to radially compress at least a portion of venous valve 504, as well as the portion of penile vein 500 that has become attached to venous valve 504. In the radially compressed configuration shown in FIG. 6, blood flow lumen 508 is substantially narrowed or closed to restrict blood flow B_{f} through venous valve 508 and thereby prevent or restrict blood outflow from the penis through penile vein 500. While venous valve 504 is radially compressed or slowly returning to the radially expanded configuration, blood flow exiting the penis through penile vein 500 will be restricted thus helping to maintain the penile erection. In an embodiment, blood flow may be restricted or prevented from flowing out of the penis via penile vein 500 for a time period of 20 to 60 minutes due to the delayed elastic recovery of venous valve 504.

Penile vein 500 extends within the pendant portion of the penis and is susceptible to finger pressure exerted on the penis by the user, and accordingly may be one of the superficial or deep dorsal veins of the penis. As noted above, venous valve 504 is intended to be positioned at a location along penile vein 500 such that venous valve 504 may be radially compressed within penile vein 500 by a user pressing firmly on or near the base of the penis, i.e., the portion of the penis that is positioned external or outside of the pubic bone and the urogenital diaphragm. In conjunction with the normal parasympathetic nervous system processes associated with arousal, venous valve 504 may be selectively compressed by a user whenever an erection is desired to be attained or maintained. As such, recoil delay component 514 is expected to be capable of repeated/numerous uses over the lifetime of the user in delaying the recoil of stent framework 506 after being subjected to compressions of venous valve 504.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A venous valve for implantation in and selectively restricting the outflow of blood from a penile vein to aid in achieving an erection, the venous valve comprising:
a self-expanding stent framework defining a blood flow lumen that extends between a first end and a second end thereof, wherein the self-expanding stent framework is adapted to recoil from a radially compressed configuration in which the blood flow lumen is narrowed to restrict blood flow through the venous valve to a radially expanded configuration in which the blood flow lumen is fully open to permit unrestricted blood flow through the venous valve; and
a recoil delay component attached to the self-expanding stent framework for slowing the recoil of the self-expanding stent framework, wherein the recoil delay component is adapted to delay the recoil of the self-expanding stent framework from the radially compressed configuration to the radially expanded configuration and thereby is adapted to provide an extended time period during which the blood flow lumen is narrowed such that blood flow through the venous valve is restricted.

2. The venous valve of claim 1, wherein the recoil delay component comprises a viscoelastic polymeric material that covers at least a portion of the stent framework.

3. The venous valve of claim 2, wherein struts of the self-expanding stent framework are encased within the viscoelastic polymeric material.

4. The venous valve of claim 3, wherein the viscoelastic polymeric material covers interstitial spaces between adjacent struts of the self-expanding stent framework.

5. The venous valve of claim 2, wherein the recoil delay component extends along an intermediate section of the self-expanding stent framework such that a first end section and a second end section of the self-expanding stent framework remain uncovered.

6. The venous valve of claim 5, wherein the first and second end sections of the self-expanding stent framework are adapted to recoil from the radially compressed configuration to the radially expanded configuration more quickly than the intermediate section of the self-expanding stent framework that is dampened by the viscoelastic polymeric material.

7. The venous valve of claim 2, wherein the self-expanding stent framework is formed of a superelastic material.

8. The venous valve of claim 1, wherein the extended time period during which the blood flow lumen is narrowed and blood flow through the venous valve is restricted is between 20 and 60 minutes.

9. The venous valve of claim 1, wherein the recoil delay component comprises a slow elastic recovery hydrogel that covers at least a portion of the self-expanding stent framework.

10. A method of using an implantable venous valve for selectively restricting the outflow of blood from a penile vein to aid in achieving an erection of a penis, the method comprising the steps of:
implanting a venous valve into a penile vein at a target location that is susceptible to a compressive radial force exerted on the penis, wherein the venous valve includes a self-expanding stent framework defining a blood flow lumen therethrough and a recoil delay component attached to the self-expanding stent framework to delay a recoil of the self-expanding stent framework from a radial compressed configuration to a radially expanded configuration; and
firmly pressing on the penis to impart a compressive radial force on the venous valve implanted at the target location thereby radially compressing the self-expanding stent framework and narrowing the blood flow lumen to restrict blood outflow from the penile vein.

11. The method of claim 10, wherein the delayed recoil of the self-expanding stent framework provides an extended time period during which the blood flow lumen of the venous valve is narrowed and blood outflow from the penile vein is restricted.

12. The method of claim 11, wherein the extended time period is between 20 and 60 minutes.

13. The method of claim 12, wherein the recoil delay component comprises a viscoelastic polymeric material that covers at least a portion of the stent framework and the stent framework is formed of a superelastic material.

14. The method of claim 10 further comprising:
releasing the compressive radial force, wherein after the compressive radial force is released the self-expanding stent framework slowly recoils to the radially expanded configuration such that blood outflow through the penile vein continues to be restricted for between 20 and 60 minutes.

15. The method of claim 10, wherein the step of implanting comprises tracking a catheter-based delivery system with the venous valve compressed within a distal end thereof through the vasculature to the target location and deploying the venous valve at the target location within the penile vein.

16. The method of claim 15, wherein the penile vein is selected from the group consisting of the deep dorsal vein and the superficial dorsal vein.

17. The method of claim 10, wherein the step of implanting includes means for keeping the venous valve at the target location while the compressed self-expanding stent framework recoils to the radially expanded configuration.

18. The method of claim 17, wherein the means for keeping the venous valve at the target location includes applying a compression bandage or external clamp/ring around the penis adjacent to and downstream of the target location.

19. The method of claim 15, wherein the step of implanting includes releasing the venous valve from the catheter-based delivery system and then expanding a balloon of a balloon catheter within the venous valve until the venous valve is in apposition with a wall of the penile vein.
